SEQUENCE LISTING (1 sequence)

SEQ ID NO: 1 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

(19) United States
(12) Patent Application Publication
(10) Pub. No.: US 2022/0204954 A1
(43) Pub. Date: Jun. 30, 2022

(54) ENGINEERED CAS9 WITH BROADENED DNA TARGETING RANGE

(71) Applicant: THE BOARD OF TRUSTEES OF THE LELAND STANFORD JUNIOR UNIVERSITY, Stanford, CA (US)

(72) Inventor: Le Cong, Stanford, CA (US)

(21) Appl. No.: 17/605,932

(22) PCT Filed: Apr. 24, 2020
(86) PCT No.: PCT/US2020/029855

§ 371 Date: Oct. 22, 2021

Related U.S. Application Data
(60) Provisional application No. 62/838,498, filed on Apr. 25, 2019.

(51) Int. Cl. C12N 9/22; C12N 15/90

(52) U.S. Cl. CPC C12N 9/22; C12N 2310/20; C12N 15/907

(57) ABSTRACT

The disclosure provides variant Staphylococcus aureus Cas9 (SaCas9) proteins with altered specificity for protospacer adjacent motif (PAM) sequences. The disclosure also is directed to CRISPR/Cas9 systems and methods of altering a genomic DNA sequence using the variant SaCas9 protein. Methods of generating variant Cas9 proteins with altered PAM specificity are also disclosed.

### CROSS-REFERENCE TO RELEATED APPLICATIONS

This application claims the benefit of U.S, provisional patent application number 62./838,498, filed Apr. 25, 2019, which is incorporated herein by reference in its entirety.

### FIELD

The present invention related to engineered CAS9 proteins with broadened DNA targeting ranges as well as methods, kits, compositions, and system employing the same.

### BACKGROUND

The Clustered Regularly Interspaced Short Palindromic Repeats (CRISPR) system, originally found in bacteria and archaea, can adaptively resist foreign genetic materials to provide microbial immunity employing RNA-guided protein machineries and intricate molecular mechanisms (Mojica et al., J Mol. Evol., 60: 174-182 (2005); Bolotin et al., Microbiology, 151: 2551-2561 (2005); Barrangou et al., Science. 315: 1709-1712 (2007); Garneau et al., Nature, 468: 67 (2010); Deltcheva et al., Nature, 471: 602 (2011); Sapranauskas et al., Nucl. Acids Res., 39: 9275-9282 (2011); Jinek et al., Science, 337: 816-821 (2012); Gasiunas et al., Proc. Natl. Acad. Sci. USA, 109: E2579-E2586 (2012); and Wiedenheft et al., Nature, 482: 331 (2012)). Recent advances enable harnessing of customized CRISPR systems for genome editing in eukaryotic organisms (Gong et al., Science, 339: 819-823 (2013); Mali et al., Science, 339: 823-826 (2013); Jiang et al., Nature Biotech., 31: 233-239 (2013); Jinek et al., Elife, 2: e00471 (2013); Cho et al., Nature Biotech., 31: 230 (2013); and Hwang et al., Nature Biotech., 31: 227 (2013)). The exemplary Type II CRISPR system employ a Cas9 protein in complex with single-guide RNA (sgRNA), forming a programmable endonuclease that cleaves a double-stranded DNA (dsDNA) target. The dsDNA substrate contains a target strand complimentary to the guide sequence in sgRNA (Jinek et al., Science, 337: 816-821 (2012)) and a non-target strand bearing a protospacer adjacent motif (PAM) required for target recognition (Mojica et al., J. Mol. Evol., 60: 174-182 (2005); Bolotin et al., Microbiology, 151: 2551-2561 (200:5)).

The widely used Cas9 from Streptococcus pyogenes (SpCas9) recognizes the PAM sequence NGG (Jinek et al., supra), while the newly identified Cas9 from Staphylococcus aureus (SaCas9) recognizes the longer PAM sequence of NNGRRT (Ran et al., Nature, 520:186-191 (2015)). SaCas9 is significantly smaller than SpCas9, making its delivery more convenient and efficient for gene therapy applications (Ran et al., supra). Despite the promise for clinical translation due to its compact size, the longer PAM of SaCas9 limits its targeting range and application potential, e.g., when its PAM is not in proximity of disease-relevant loci. Recently, a set of triple mutations E782K/N968K/R1015H (KKH) was found to effectively alter SaCas9 PAM specificity from NNGRRT to NNNRRT (Kleinstiver et al., Nature Biotech., 33: 1293-1298 (2015)). In addition, the structure of wild-type SaCas9 bound with sgRNA/DNA has been resolved (Nishimasu et al., Cell, 162: 1113-1126 (2015)), which provides valuable insights on the molecular basis of SaCas9 function.

However, there remains a need for Cas9 proteins with broader PAM specificity, as well as methods for altering PAM specificity of Cas9 proteins.

### SUMMARY

The disclosure provides variant Staphylococcus aureus Cas9 (SaCas9) proteins comprising, for example, the amino acid sequence of SEQ ID NO: 1, wherein one or more of amino acid residues E782, N968, N986, and R991 are substituted with a different amino acid. Also provided are nucleic acid sequences and vectors encoding the variant SaCas9 protein, as well as systems and methods for altering a target genomic DNA sequence in a host cell.

The disclosure also provides methods of generating a variant Cas9 protein with a desired PAM specificity, which method comprises: (a) molecularly simulating binding of one or more mutant Cas9 proteins to a desired PAM; (b) synthetically generating one or more mutant Cas9 proteins that bind to the desired PAM in the simulation of (a), (c) expressing the one or more mutant Cas9 proteins in a host cell in combination with a guide RNA sequence that is complementary to a target DNA sequence in the host cell, wherein the host cell genome comprises the target DNA sequence and the desired PAM; (d) measuring the cleavage activity of the one more mutant Cas9 proteins; and (e) selecting one or more mutant Cas9 proteins which bind to the desired PAM and cleave the target DNA sequence, whereby a variant Cas9 with a desired PAM specificity is generated.

Further provided are kits containing one or more reagents or other components useful, necessary, or sufficient for practicing any of the methods described herein. For example, kits may include CRISPR reagents (Cas9 protein, guide sequences, plasmids, etc.), transfection or administration reagents, negative and positive control samples (e.g., cells, template DNA), cells, containers housing one or more components (e.g., microcentrifuge tubes, boxes), detectable labels, detection and analysis instruments, software, instructions, and the like.

### BRIEF DESCRIPTION OF THE DRAWINGS)

FIG. 1A is a schematic diagram illustrating a model system for MID simulations of SaCas9 with bound DNA and RNA. The interaction between the PAM region of DNA and its surrounding protein residues is enlarged. FIG. 1B is a graph showing time-dependent distances for pairs E782-K910 and E782-G0. FIG. 1C is a graph showing time-dependent distances for pairs N968-G3 and R1015-G3. FIG. 1D is a series of images showing coordinations of E782 at 0, 57 and 80 ns. In FEP calculations for the E782K mutation, the Na+ ion in FIG. 1D was annihilated along with E782 to avoid its unfavorable clash (or electrostatic repulsion) with the emerging the K782. Accordingly, in FEP calculations for the free state, an extra Na+ in the electrolyte (not close to the protein complex) was annihilated simultaneously with the E782.

FIGS. 2A-2C are schematic diagrams of atomic structures of the crystallized SaCas9 complex. FIG. 2A shows a unit cell comprising four complex copies, labelled as A, B, C and D. FIG. 2B shows an enlarged view of the crystal contact between copies A and B, or C and D. FIG. 2C shows an enlarged view of the crystal contact between copies B and C.

FIGS. 3A-3C are schematic diagrams of MD simulation of the SaCas9 complex. FIG. 3A shows root-mean-square-deviations (RMSD) of the protein, DNA, and RNA backbones during the about 200 ns simulation. FIG. 3B shows atomic coordinations between the PI domain in SaCas9 and the PAM in the non-target DNA strand. FIG. 3C shows the overlap of the crystal structure (grey) and the final simulate structure (green).

FIG. 4 is a graph illustrating root-mean-square-deviations of protein backbones in MD simulation on SaCas9 with the bound sgRNA only. Inset: A snap shot of the complex in the end of MD simulation.

FIG. 5A is a series of schematic: diagrams showing the thermodynamic cycle for calculating ΔΔG of the mutation R1015H. ΔG_{A} and ΔG_{B} are free energy changes for the dsDNAs binding to the wide-type and mutant proteins, respectively; ΔG₁ and ΔG₂, are free energy changes for the mutation occurring at the DNA-bound state and DNA-free state, respectively. Atoms in protein residues 993 and 1015 are highlighted as van der Waals spheres. FIG. 5B is a graph illustrating free energy changes of alanine scanning on selected residues involved in PAM recognition. FIG. 5C is a graph illustrating normalized Cas9 efficiency as measured in mammalian cell experiments using molecular constructs corresponding to the mutation scanning performed in computational analysis. FIG. 5D is a graph showing robust linear correlation between FEP results and experimental Cas9 efficiency demonstrating validity of the COMET workflow. Linear regression was performed using ΔΔG and natural log (ln) of efficiency ratio for each mutant Cas9 tested over wild-type control. Goodness of fit by R square was 0.92.

FIG. 6A is a graph illustrating free energy changes for various mutations associated with the KKH SaCas9 mutant. FIG. 6B is a schematic diagram illustrating the E782K mutation in SaCas9. Atoms in protein residues K782 and K910 are highlighted as van der Waals spheres, FIG. 6C is a schematic diagram illustrating the role of water in E782K and N968K. mutations. FIG. 6D is a schematic diagram of a perspective view of key interactions between KKH-SaCas9 protein and bound DNA.

FIG. 7A is a graph showing FEP calculations for various mutations for COMET-based optimization of SaCas9 variants with expanded PAM range. FIG. 7B is a graph showing normalized Cas9 efficiency for engineered saCas9 variants targeting NNGRRT=C=G=A PAM. FIG. 7C is a schematic illustration of R986's coordination with the DNA backbone and the hydrophobic interaction between 8986 and L991 (after the R991L mutation). FIG. 7D is graph showing endogenous genome targeting activity of novel SaCas9 variants discovered through COMET workflow, dash line represents wild type SaCas9 activity as the basis for normalization. For each PAM sequence shown on the X-axis, results from different targets were represented with S.E.M. as error bar. FIG. 7E is a diagram summarizing COMET for a combined approach to understand and engineer CRISPR genome editing tools.

FIG. 8 is a graph illustrating experimental verification and characterization of SaCas9 variants bearing N986R and additional R991 combinatorial mutations to further enhance its targeting range, shown are Cas9 efficiency for SaCas9 variants normalized to wild type SaCas9. Different color bards represents targets bearing different PAM sequences where its last position is varied to include all four DNA bases.

FIGS. 9A-9D are graphs illustrating activities of different SaCas9 variants compared with wild type SaCas9 over different PAM sequence groups, detailing individual genomic sites tested in the assay. Each data bar represents results from independent replicates and error bars showing S.E.M.

FIG. 10 is a schematic diagram of a structural analysis for additional residues of SaCas9 enhancing PAM recognition.

FIG. 11 is a graph illustrating Cas9 activity of SaCas9 variants with combinations of mutations focusing on recognizing the PAM duplex on target DNA. Results are colored by binding to DNA targets with different PAM sequences,

FIG. 12 is a graph illustrating Cas9 activity of SaCas9 variants with combinations of mutations focusing on enhancing general binding affinity of target DNA. Results are colored by binding to DNA targets with different PAM sequences.

FIG. 13 is a graph illustrating Cas9 activity of SaCas9 variants as measured by cleavage of genomic targets, and that cutting activity on target DNA would be different from the binding activity as measured in FIG. 7. The different colors represent results from cleaving DNA targets with different PAM sequences.

### DETAILED DESCRIPTION OF THE INVENTION

The present disclosure is predicated, at least in part, on the development of a method combining computational analysis and experimental assay to identify variant Cas9 proteins with altered PAM specificity. In particular, the disclosed methods enable the design of variant SaCas9 proteins that harbor expanded PAM activities for gene editing for sequences that were previously non-targetable. The methodology described herein may serve as a general motif in exploring non-natural CRISPR utilities combining the power of computational physical chemistry and gene editing.

### Definitions

To facilitate an understanding of the present technology, a number of terms and phrases are defined below. Additional definitions are set forth throughout the detailed description.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA. helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60%, (e.g., 65%, 70%, 75%, 80%, 85%, 90%, 95%, 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardes solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50%(v/v) formamide with 0.1% bovine serum albumin (BSA)/0.1% Ficoll/0.1% polyvinylpyrrolidone (PVP)/50 nM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46: 453 (1960) and Doty et al., Proc. Natl. Acad. Sci. USA, 46: 461 (1960), have been followed by the refinement of this process into an essential tool of modern biology. For example, hybridization and washing conditions are now well known and exemplified in Sambrook, J. Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell. W., Molecular Cloning: A Laboratory Manual, Third Edition, Cold Spring Harbor Laboratory Press, Cold Spring Harbor (2001). The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) comprises a “double-stranded nucleic acid.” For example, triplex structures are considered to be “double-stranded.” In some embodiments, any base-paired nucleic acid is a “double-stranded nucleic acid.”

The term “gene” refers to a DNA sequence that comprises control and coding sequences necessary for the production of an RNA having a non-coding function (e.g., a ribosomal or transfer RNA), a polypeptide, or a precursor. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or a RNA chain that has functional role to play in an organism. For the purpose of this disclosure it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “variant” refers to the exhibition of qualities that have a pattern that deviates from what occurs in nature. In some embodiments, a variant may also be a mutant.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “oligonucleotide” as used herein is defined as a molecule comprising two or more deoxyribonucleotides or ribonucleotides, preferably at least 5 nucleotides, more preferably at least about 10 to 15 nucleotides and more preferably at least about 15 to 50 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotides). The exact size will depend on many factors, which in turn depend on the ultimate function or use of the oligonucleotide, The oligonucleotide may be generated in any manner, including chemical synthesis, DNA replication, reverse transcription, PCR, or a combination thereof.

The terms “peptide” and “polypeptide” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g., with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein binding protein). In the case of a protein domain-binding protein, it can bind to itself (to form homodimers, homotrimers, etc.) and/or it can bind to one or more molecules of a different protein or proteins.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms). Alternatively, DNA sequences encoding RNA (e.g., DNA-targeting RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert,” may be attached or incorporated so as to bring about the replication of the attached segment in a cell.

A cell has been “genetically modified,” “transformed,” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

CRISPR/Cas gene editing systems have been developed to enable targeted modifications to a specific gene of interest in eukaryotic cells. CRISPR/Cas gene editing systems are based on the RNA-guided Cas9 nuclease from the type II prokaryotic clustered regularly interspaced short palindromic repeats (CRISPR) adaptive immune system (see, e.g., Jinek et al., Science, 337: 816 (2012); Gasiunas et al., Proc. Natl. Acad. Sci. U.S.A., 109, E2579 (2012); Garneau et al., Nature, 468: 67 (2010); Deveau et al., Annu. Rev. Microbial., 64: 475 (2010); Horvath and Barrangou, Science, 327: 167 (2010); Makarova et al., Nat. Rev. Microbial., 9, 467 (2011); Bhaya et al., Annu. Rev. Genet., 45: 273 (2011); and Cong et al., Science, 339: 819-823 (2013)). In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Each CRISPR locus encodes acquired “spacers” that are separated by repeat sequences. Transcription of a CRISPR locus produces a “pre-crRNA,” which is processed to yield crRNAs containing spacer-repeat fragments that guide effector nuclease complexes to cleave dsDNA sequences complementary to the spacer.

The type II CRISPR locus comprises four genes, including the gene encoding the Cas9 protein, two noncoding crRNAs: trans-activating crRNA (tracrRNA) and a precursor crRNA (pre-crRNA) array containing nuclease guide sequences (also referred to as “spacers”) interspaced by identical direct repeats (DRs) (Cong et al., supra). tracrRNA is important for processing the pre-crRNA and formation of the Cas9 complex. CRISPR-guided degradation of pathogenic sequences occurs in three steps. First, tracrRNAs hybridize to repeat regions of the pre-crRNA. Second, endogenous RNaseIII cleaves the hybridized crRNA-tracrRNAs, and a second event removes the 5′ end of each spacer, yielding mature crRNAs that remain associated with both the tracrRNA and Cas9. Third, each mature complex locates a target double stranded DNA (dsDNA) sequence and cleaves both strands.

Engineering CRISPR/Cas systems for use in eukaryotic cells typically involves reconstitution of the crRNA-tracrRNA-Cas9 complex. In human cells, for example, the Cas9 amino acid sequence may be codon-optimized and modified to include an appropriate nuclear localization signal, and the crRNA and tracrRNA sequences may be expressed individually or as a single chimeric molecule via an RNA polymerase II promoter. Typically, the crRNA and tracrRNA sequences are expressed as a chimera, and are referred to collectively as “guide RNA” (gRNA) or single guide RNA (sgRNA). Thus, the terms “guide RNA,” “single guide RNA,” and “synthetic guide RNA,” are used interchangeably herein and refer to a nucleic acid sequence comprising a tracrRNA and a pre-crRNA array containing a guide sequence. The terms “guide sequence,” “guide,” and “spacer,” are used interchangeably herein and refer to the about 20 nucleotide sequence within a guide RNA that specifies the target site. In CRISPR/Cas9 systems, the guide RNA contains a 2.0 nucleotide guide sequence followed by a protospacer adjacent motif (PAM) that directs Cas9 via Watson-Crick base pairing to a target sequence (Deveau et al., Annu. Rev. Microbiol., 64: 475-493 (2010); Jinek et al., Science, 337: 816-821 (2012); and Xie et al., Genome Res., 24(9): 1526-1533 (2014)). Canonical PAM sequences are NGG or NAG for Cas9 from Streptococcus pyogenes and NNNNGATT for the Cas9 from Neisseria meningitidis.

The disclosure provides variant Cas proteins. The variant Cas protein may be based on or derived from any suitable Cas protein (or homolog or modified version thereof). Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, Cas protein families are described in further detail in, e.g., Haft et al., PLoS Comput. Biol., 1(6): e60 (2005). In one embodiment, the variant Cas protein is based on or derived from a wild-type Cas9 protein. The Cas9 protein can be obtained from any suitable microorganism, and a number of bacteria express Cas9 protein variants. The Cas9 from Streptococcus pyogenes and S. thermophilus are widely used in the art; however, other Cas9 proteins have high levels of sequence identity with the S. pyogenes Cas9 and use the same guide RNAs. Cas9 proteins of other species are known in the art (see, e.g., U.S. Patent Application Publication 2017/0051312) and may be used in connection with the present disclosure. The Cas9 protein is further described in, e.g., Mali et al., Nat Methods, 10(10): 957-963 (2013), and the amino acid sequences of Cas proteins from a variety of species are publicly available through the GenBank and UniProt databases.

In one embodiment, the variant Cas9 protein is obtained or based upon a Staphylococcus aureus Cas9 (SaCas9) protein, ideally a wild-type S. aureus Cas9 protein. SaCas9 was recently identified in a search for Cas9 orthologs that are small, efficient, and broadly targeting, making their delivery more convenient and efficient for gene therapy applications (Ran et al., Nature, 520(7546): 186-191 (2015)). SaCas9 achieves the highest known editing efficiency in mammalian cells with guide RNA sequences between 21- to 23-nt long and can accommodate a range of lengths for the direct repeat:anti-repeat region. Although SaCas9 cleaves genomic targets most efficiently via a PAM sequence of NNGRRT, all NNGRR PAMs can be cleaved by SaCas9 (Ran et al., supra; and Friedland et al., Genome Biology, 16: 257 (2015)). Exemplary wild-type SaCas9 amino acid sequences include the amino acid sequence deposited in the UniProt database under Accession No. J7RUAS (CAS9_STAAU), and SEQ ID NO: 1. Plasmids comprising nucleic acid sequences encoding SaCas9 are publicly available from the Addgene repository.

In one embodiment, the variant SaCas9 protein comprises the amino acid sequence of SEQ ID NO: 1, but further comprises a substitution of one or more amino acid residues of SEQ ID NO: 1. An amino acid “replacement” or “substitution” refers to the replacement of one amino acid at a given position or residue by another amino acid at the same position or residue within a polypeptide sequence, Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained.

“Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

The variant SaCas9 protein may comprise, consist essentially of, or consist of any one, or combination of, suitable amino acid substitutions of SEQ ID NO: 1, so long as the variant SaCas9 retains the useful activity of the parent SaCas9 protein, or more preferably, exhibits enhanced activity or properties as compared to the parent protein (e.g., nuclease activity, the ability to interact with a guide RNA and target DNA, etc.). In one embodiment, the variant SaCas9 protein comprises the amino acid sequence of SEQ ID NO: 1, except that one or more of amino acid residues E782, N968, N986, and R991 are substituted with a different amino acid. The amino acids of these positions may each individually be modified or combinations may be modified (e.g., positions 986 and 991, positions 968 and 986, positions 782 and 986, positions 782, 986, and 991, positions 968, 986, and 991 are modified). The asparagine residue at position 986 of SEQ ID NO: 1 may be substituted with any suitable amino acid residue, such as, for example, alanine (N986A), arginine (N986R), lysine (N986K), or histidine (N986H). Similarly, the arginine residue at position 991 of SEQ ID NO: 1 may be substituted with any suitable amino acid residue, such as, for example, alanine (R991A), lysine (R991K), leucine (R991L), cysteine (R991C), or valine (R991V). The glutamic acid residue at position 782 of SEQ ID NO: 1 may be substituted with any suitable amino acid residue, such as, for example, lysine (E782K), arginine (E782R), or histidine (E782H). The asparagine residue at position 968 of SEQ ID NO: 1 may be substituted with any suitable amino acid residue, such as, for example, lysine (N968K), arginine (N968R), or histidine (N968H).

In some embodiments, the variant SaCas9 protein may further comprise an amino acid substitution of one or more residues of SEQ ID NO: 1 selected from N885 (asparagine, Asn), K886 (lysine, K), L887 (leucine. L), N888 (asparagine, Asn), A889 (alanine, Ala), 81015 (arginine, Arg), and T1019 (threonine, Thr). The asparagine residue at position 885 of SEQ ID NO: 1 may be substituted with any suitable amino acid residue, such as, for example, lysine (N885K). The lysine residue at position 886 of SEQ m NO: 1 may be substituted with any suitable amino acid residue, such as, for example, asparagine (K886N) or arginine (K886R). The lysine residue at position 887 of SEQ ID NO: 1 may be substituted with any suitable amino acid residue, such as, for example, leucine (L887K). The lysine residue at position 888 of SEQ ID NO: 1 may be substituted with any suitable amino acid residue, such as, for example, asparagine (N888K). The alanine residue at position 889 of SEQ ID NO: 1 may be substituted with any suitable amino acid residue, such as, for example, histidine (A889H), lysine (A889K), or asparagine (A889N). The arginine residue at position 1015 of SEQ ID NO: 1 may be substituted with any suitable amino acid residue, such as, for example, histidine (R1015H). The threonine reside at position 1019 of SEQ ID NO: 1 may be substituted with any suitable amino acid residue, such as, for example, arginine (T1019R), lysine (T1019K), or histidine (T1019H).

The variant SaCas9 protein may comprise, consist essentially of, or consist of any one or combination of the above-described amino acid substitutions of SEQ ID NO: 1. In some embodiments, the variant Cas9 protein comprises the amino acid sequence of SEQ ID NO: 1 and two or more (e.g., 2, 3, 4, 5, or more) amino acid substitutions. For example, the variant SaCas9 protein may comprise, consist essentially of, or consist of substitution of two amino acid residues of SEQ ID NO: 1, including but not limited to, N986R and R991A; N986R and R991K; N986R and R991L; N885K and N986R; K886N and N986R; K886R and N986R; L887K and N986R; N888K and N986R; A889H and N986R; A889K and N986R; A889N and N986R; E782K and N986R; N968K and N986R; E782K and N986R; N968K and N986R, or any other combination of two of the foregoing substitutions. In other embodiments, the variant SaCas9 protein may comprise, consist essentially of, or consist of substitution of three amino acid residues of SEQ ID NO: 1, including but not limited to, N986R, R991A, and T1019R, N986R, R991A, and T1019K; N986R, R991A, and T1019H; N986R, R991K, and T1019R; N986R, R991K, and T1019K; N986R, R991K, and T1019H; N986R, R991L, and T1019R; N986R, R991L, and T1019K; N986R, R991L, and T1019H; N986R, R991C, and T1019R; N986R, R991C, and T1019K; N986R, R991C, and T1019H; N986R, R991V, and T1019R; N986R, R991V, and T1019K; N986R, R991V, and T1019H; N885K, N986R, and R991L; K886N, N986R, and R991L; K886R, N986R, and R991L; L887K, N986R, and R991L; N888K, N986R, and R991L; A889H, N986R, and R991L; A889K, N986R, and R991L; A889N, N986R, and R991L; E782K, N968K, and N986R; E782K, N986R, and R1015H; N968K, N986R, and R1015H; E782K, N986R, and R991L; N968K, N986R, and R991L; or any combination of three the foregoing substitutions. In other embodiments, the variant SaCas9 protein may comprise, consist essentially of, or consist of substitution of four amino acid residues of SEQ ID NO: 1, including but not limited to, E782K, N968K, N986R, and R1015H; E782K, N968K, N986R, and R991L; E782K, N986R, R991L, and R1015H; N968K, N986R, R991L, and R1015H, or any combination of four of the foregoing substitutions. In some embodiments, the variant SaCas9 protein may comprise, consist essentially of, or consist of substitution of five amino acid residues of SEQ ID NO: 1, including but not limited to E782K, N⁻968K, N986R, R991L, and R1015H, or any combination of five of the foregoing substitutions. Variant SaCas9 proteins comprising more than five amino acid substitutions (e.g., 6, 7, 8, 9, 10 or more substitutions) also are within the scope of the present disclosure.

In some embodiments, the disclosure provides a Cas9 protein which comprises an amino acid sequence that is at least 90% identical (e.g., at least 91%, at least 92%, at least 93%, at least 94%, at least 95%, at least 96%, at least 97%, at least 98%, at least 99%, or 100% identical) to SEQ ID NO: 1, with or without any of amino acid substitutions described herein. Nucleic acid or amino acid sequence identity can be determined by comparing a nucleic acid or amino acid sequence of interest to a reference nucleic acid or amino acid sequence, as described herein.

The present disclosure also provides an isolated or purified nucleic acid sequence encoding the variant SaCas9 protein described herein. Also provided is a vector comprising the isolated nucleic acid, optionally operably linked to one or more expression control sequences (e.g., promoters, enhancers, polyadenylation signals, transcription terminators, internal ribosome entry sites (IRES), and the like) that provide for the expression of the nucleic acid sequence in a host cell (e.g., a mammalian cell). The vector can be, for example, a plasmid, episome, cosmid, viral vector (e.g., retrovirus, adenovirus, lentivirus, or adeno-associated virus vector), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)). Exemplary expression control sequences for control of gene expression in vector systems include prokaryotic and eukaryotic sequences described in, for example, Goeddel, Gene Expression Technology; Methods in Enzymology, Vol. 185, Academic Press, San Diego, Calif. (1990), Sambrook et al,, supra; and Ausubel et al., supra.

The choice of expression control sequences, such as promoters, depends on the particular application of the vector and systems described herein. A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, Calif), LACSWITCH™ system (Stratagene, San Diego, Calif.), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 3086: 123-144 (2005)).

A nucleic acid sequence encoding the variant SaCas9 protein can be provided to a cell on the same vector (i.e., in cis) as a cognate guide RNA sequence (sgRNA). In such embodiments, a unidirectional promoter can be used to control expression of each nucleic acid sequence. In another embodiment, a combination of bidirectional and unidirectional promoters can be used to control expression of multiple nucleic acid sequences. In other embodiments, a nucleic acid sequence encoding the variant SaCas9 protein and its cognate guide RNA sequence can be provided to a cell on separate vectors (i.e., in trans). Each of the nucleic acid sequences in each of the separate vectors can comprise the same or different expression control sequences, The separate vectors can be provided to cells simultaneously or sequentially.

A vector comprising the nucleic acid sequence encoding a variant SaCas9 protein can be introduced into a host cell that is capable of expressing the polypeptide encoded thereby, including any suitable prokaryotic or eukaryotic cell, As such, the disclosure provides an isolated cell comprising the vector or nucleic acid sequences disclosed herein. Preferred host cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif) and are described in, for example, Kitts et al., Biotechniques, 14: 810-817 (1993); Lucklow, Curr. Opin. Biotechnol., 4: 564-572 (1993); and Lucklow et al., J. Viral., 67: 4566-4579 (1993). Desirably, the host cell is a mammalian cell, and in some embodiments, the host cell is a human cell. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of cells are known in the art.

The disclosure provides CRISPR/Cas systems comprising the variant SaCas9 proteins described herein. As used herein, “CRISPR/Cas system” refers collectively to transcripts and other elements involved in the expression of and/or directing the activity of CRISPR-associated (“Cas”) genes, including sequences encoding a Cas gene, Cas protein, a tracr (trans-activating CRISPR) sequence (e.g., tracrRNA or an active partial tracrRNA), a cr (CRISPR) sequence (e.g., crRNA or an active partial crRNA), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Staphylococcus aureus or Streptococcus pyogenes. In certain embodiments, the Cas9 protein can be included in the system separate from, associated with, or encoded by, a vector. Thus, the disclosure provides system comprising: (a) a guide RNA sequence that is complementary to a target genomic DNA sequence in a host cell, wherein the target genomic DNA sequence encodes at least one gene product; and (b) a nucleic acid molecule comprising a nucleic acid sequence encoding the variant SaCas9 protein described herein. In other embodiments, the disclosure provides a system comprising (a) a guide RNA sequence that is complementary to a target genomic DNA sequence in a host cell, wherein the target genomic DNA sequence encodes at least one gene product; and (b) the variant SaCas9 protein described herein. When the system comprises a guide RNA sequence and a nucleic acid sequence encoding the variant SaCas9 protein, the guide RNA sequence of and the nucleic acid molecule encoding the variant SaCas9 protein may be present in different vectors or present in the same vector, as discussed above. When the Cas9 protein is included in the system separate from the vector, it is desirably included in a single composition (e.g., a pharmaceutical composition) alone or in combination with a vector comprising the guide RNA sequence, and is not physically or chemically bound to the vector. In other embodiments, the Cas9 protein may be “associated” with a vector comprising the guide RNA sequence if it is physically or chemically linked or bound to the vector, such that a complex between the Cas9 protein and vector is formed (e.g., a complex between the Cas9 protein and a viral vector). The Cas9 protein can be associated with a vector using any suitable method for protein-protein linking or protein-virus linking known in the art.

The terms “target sequence,” “target nucleic acid,” and “target site” (e.g., a “target genomic DNA sequence”) are used interchangeably herein to refer to a polynucleotide (nucleic acid, gene, chromosome, genome, etc.) in a host cell to which a guide sequence (e.g., a guide RNA) is designed to have complementarity, wherein hybridization between the target sequence and a guide sequence promotes the formation of a CRISPR complex, provided sufficient conditions for binding exist. The term “genomic,” as used herein, refers to a nucleic acid sequence (e.g., a gene or locus) that is located on a chromosome in a cell. The target sequence and guide sequence need not exhibit complete complementarity, provided that there is sufficient complementarity to cause hybridization and promote formation of a CRISPR complex. A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art; see, e.g., Sambrook, referenced herein and incorporated by reference. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “noncompletnentary strand” or “non-complementary strand.”

The target genomic DNA sequence desirably encodes a gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target genomic DNA sequence encodes a protein or polypeptide.

The disclosure also provides a method of altering a target genomic DNA sequence in a host cell, which method comprises contacting a host cell comprising a target genomic DNA sequence with the systems described herein, wherein: (a) the guide RNA sequence is expressed in the host cell and binds to the target genomic DNA sequence in the host cell genome, (b) the variant SaCas9 protein is expressed in the host cell and induces a double strand break in the target genomic DNA sequence, thereby altering the target genomic DNA sequence in the host cell. Descriptions of the variant SaCas9 protein, the guide RNA sequence, the host cell, the target genomic DNA sequence, and components thereof, set forth above in connection with the inventive system also are applicable to the method of altering a target genomic DNA sequence in a host cell.

The phrase “altering a DNA sequence,” as used herein, refers to modifying at least one physical feature of a wild-type DNA sequence of interest. DNA alterations include, for example, single or double strand DNA breaks, deletion or insertion of one or more nucleotides, and other modifications that affect the structural integrity or nucleotide sequence of the DNA sequence, In one embodiment, the method introduces a single strand or double strand break in the target DNA sequence. In this respect, the variant SaCas9 protein directs cleavage of one or both strands of a target DNA sequence, such as within the target genomic DNA sequence and/or within the complement of the target sequence. In some embodiments, the variant SaCas9 protein directs cleavage of one or bath strands of a target sequence within about 1, 2, 3, 4, 5, 8, 9, 10, 15, 20, 25, 50, 100, 200, 500, or more base pairs from the first or last nucleotide of a target sequence.

Desirably, the disclosed method alters a target genomic DNA sequence in a host cell so as to modulate expression of the target DNA sequence, i.e., expression of the target DNA sequence is increased or decreased. In one embodiment, the variant SaCas9 protein cleaves a target DNA sequence of the host cell to produce double strand DNA breaks. The double strand breaks can be repaired by the host cell by either non-homologous end joining (NHEJ) or homologous recombination. In NHEJ, the double-strand breaks are repaired by direct ligation of the break ends to one another. As such, no new nucleic acid material is inserted into the DNA break location; however, some nucleic acid material may be lost, resulting in a deletion. In homologous recombination repair, a donor nucleic acid molecule comprising a second. DNA sequence with homology to the cleaved target DNA sequence is used as a template for repair of the cleaved target DNA sequence, resulting in the transfer of genetic information from the donor nucleic acid molecule to the target DNA. As a result, new nucleic acid material is inserted/copied into the DNA break site. The modifications of the target sequence due to NHEJ and/or homologous recombination repair lead to, for example, gene correction, gene replacement, gene tagging, transgene insertion, nucleotide deletion, gene disruption, gene mutation, gene knock-down, and the like.

In some embodiments, the systems and methods described herein may be used to correct one or more defects or mutations in a gene (referred to as “gene correction”) In such cases, the target genomic DNA sequence encodes a defective version of a gene, and the CRISPR/Cas system further comprises a donor nucleic acid molecule which encodes a wild-type or corrected version of the gene. Thus, in other words, the target genomic DNA sequence is a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g, Chial, H. Rare Genetic Disorders: Learning About Genetic Disease Through Gene Mapping, SNPs, and Microarmy Data, Nature Education 1(1):192 (2008); Online Mendelian Inheritance in Man (OMIM) (www.ncbi.nim.nih.gov/entrez/query.fcgi?db=OMIM); and the Human Gene Mutation Database (HGMD) (www.hgmd.cf.ac.uk). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects.

In another embodiment, the method of altering a target genomic DNA sequence can be used to delete nucleic acids from a target sequence in a host cell by cleaving the target sequence and allowing the host cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

As discussed herein, the variant SaCas9 proteins exhibit altered and improved PAM specificity as compared to the wild-type SaCas9 protein. The altered PAM specificity enables the SaCas9 variants to efficiently disrupt genome loci that are not currently targetable. Thus, in some embodiments, the variant SaCas9 proteins are active in a host cell genome which comprises a protospacer adjacent motif (PAM) comprising the nucleic acid sequence NNGRR[T/A/C/G] located adjacent to the target genomic DNA sequence, wherein “N” is guanine, adenine, thymine, or cytosine and “R” is guanine or adenine. The PAM is “adjacent to” the target genomic DNA sequence in that it typically immediately follows the target sequence. The PAM sequence recognized by a particular variant SaCas9 protein will vary depending on the specific amino acid substitutions present in the variant. In certain embodiments, the PAM recognized by the disclosed variant SaCas9 protein comprises the nucleic acid sequence NNGRRT, NNGRRC, NNGRRA, or NNGRRG.

Any element of any suitable CRISPR/Cas gene editing system known in the art can be employed in the systems and methods described herein, as appropriate. CRISPR/Cas gene editing technology is described in detail in, for example, Cong et al., supra; Xie et al., supra; U.S. Patent Application Publication 2014/0068797; U.S. Pat. Nos. 8,697,359; 8,771,945; and 8,945,839; US2010/0076057; US2011/0189776; US2011/0223638; US2013/0130248; WO/2008/108989; WO/2010/054108; WO/2012/164565; WO/2013/098244; WO/2013/176772; US20150050699; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702, US20140242700; US20140242699; US20140242664; US20140234972; US20140227787; US20140212869; 0520140201857; US20140199767; US20140189896; US20140186958; US20140186919; US20140186843; US20140179770; US20140179006; and US20140170753; Makarova et al., Nature Reviews Microbiology, 9(6): 467-477 (2011); Wiedenheft et al., Nature, 482: 331-338 (2012); Gasiunas et al., Proceedings of the National Academy of Sciences USA, 109(39): E2579-E2586 (2012); Jinek et al., Science, 337: 816-821 (2012); Carroll, Molecular Therapy, 20(9): 1658-1660 (2012); Al-Attar et al., Biol Chem., 392(4): 277-289 (2011); and Hale et al., Molecular Cell, 45(3): 292-302. (2012).

The disclosure further provides a method of generating a variant Cas9 protein with a desired PAM specificity which combines molecular dynamics and experimental target validation. The method comprises: (a) molecularly simulating binding of one or more mutant Cas9 proteins to a desired PAM; (b) synthetically generating one or more mutant Cas9 proteins that bind to the desired PAM in the simulation of (a), (c) expressing the one or more mutant Cas9 proteins in a host cell in combination with a guide RNA sequence that is complementary to a target DNA sequence in the host cell, wherein the host cell genome comprises the target DNA sequence and the desired PAM; (d) measuring the cleavage activity of the one more mutant Cas9 proteins; and (e) selecting one or more mutant Cas9 proteins which bind to the desired PAM and cleave the target DNA sequence, whereby a variant Cas9 with a desired PAM specificity is generated.

The term “molecular dynamics (MD),” as used herein, refers to a computer simulation method for studying the physical movements of atoms and molecules. The atoms and molecules are allowed to interact for a fixed period of time, giving a view of the dynamic evolution of the system. MD simulations, complimentary to experimental studies, have proven to be effective in understanding protein-DNA interactions. (Palermo et al., Proc. Natl. Acad. S′ci. USA, 114: 7260-7265 (2017); and Cong et al., Nat. Commun., 3: 968 (2012)). Methods for probing various structural components of CRISPR/Cas9 systems, including Cas9 binding interactions with PAM sequences, have been described in the art and can be used in connection with the present disclosure (see, e.g., Estarellas et al., Biochim Biophys Acta, 1850(5): 1072-1090 (2015); Palermo et al., J Am Chem Soc., 139(45):16028-16031 (2017); Palermo et al., ACS Cent Sci., 2(10):756-76 (2016); Huai et al., Nat Conunun., 8(1):1375 (2017); and Wan et al., Sci Rep., 9(1):3188 (2019)). Detailed MD simulation methodology for the variant SaCas9 proteins described herein is set forth in the Examples. The mutant Cas9 protein may be based on or derived from any suitable wild-type Cas9 protein from any species, such as those described herein.

In some embodiments, molecularly simulating binding of one or more mutant Cas9 proteins to a desired PAM comprises free energy perturbation (FEP) calculations. The term “free energy perturbation,” as used herein, refers to a method based on statistical mechanics that is used in computational chemistry for computing free energy differences from molecular dynamics. FEP calculations have been widely used for in silica mutagenesis studies of proteins, as well as for studying host-guest binding energetics, pKa predictions, solvent effects on reactions, and enzymatic reactions. The FEP method is described in detail in, e.g., Chipot, C.; Pohorille, A. (eds.), Free Energy Calculations, Springer (2007); and Steinbrecher et al., J Mol Biol., 429(7): 923-929 (2017).

Molecular dynamic simulation allows for the identification of potential amino acid substitutions in a Cas9 protein that may alter (e.g., improve or broaden) PAM specificity. Thus, following molecularly simulating binding of one or more mutant Cas9 proteins to a desired PAM sequence; the method comprises synthetically generating one or more mutant Cas9 proteins that bind to the desired PAM sequence in the simulation of (a). The one or more mutant Cas9 proteins may be synthetically generated using recombinant DNA techniques and/or in vitro protein synthesis methods known in the art (see, e.g., Sambrook et al., supra). A wild-type Cas9 amino acid sequence can be mutated to produce Cas9 mutant by any suitable method known in the art, such as, for example, by insertion, deletion, and/or substitution. For example, mutations may be introduced into a nucleic acid sequence encoding a wild-type Cas9 protein randomly or in a site-specific manner. Random mutations may be generated, for example, by error-prone PCR of a Cas9 template sequence. Site-specific mutations can be introduced, for example, by ligating into an expression vector a synthesized oligonucleotide comprising the modified site. Alternately, oligonucleotide-directed site-specific mutagenesis procedures can be used, such as those disclosed in Walder et al., Gene, 42: 133 (1986); Bauer et al., Gene, 37: 73 (198:5); Craik, Biotechniques, 12-19 (January 1995); and U.S. Pat. Nos. 4,518,584 and 4,737,462.

In order to assess the PAM specificity of the one or more Cas9 mutant proteins predicted by the molecular dynamics simulation, the one or more mutant Cas9 proteins may be expressed in a host cell in combination with a guide RNA sequence that is complementary to a target DNA sequence in the host cell, wherein the host cell genome comprises the target DNA sequence and the desired PAM. Descriptions of the host cell, guide RNA sequence, target DNA sequence, and components thereof, set forth above in connection with the inventive system and method of altering a nucleic acid sequence also are applicable to the method of generating a variant Cas9 protein. Once expressed in the host cell, the cleavage activity of the one or more mutant Cas9 proteins can be measured using any suitable assay for measuring endonuclease activity. Such assays are described in, for example, Ander, C. and M. Jinek, Methods Enzymol., 546: 1-20 (2014); Maria J. Yebra and Ashok S. Bhagwat, Nucleic Acids Research, 21(24): 5797-5798 (1993); Zhang et al., Chem Sci., 7(8): 4951-495 (2016); and Seamon et al., Anal. Chem., 90 (11): 6913-6921 (2018). The engineering strategy described herein may be performed with any wild-type or synthetic Cas9 protein, or derivatives thereof, to further diversify the range of targetable PAMs.

The following examples further illustrate the invention but, of course, should not be construed as in any way limiting its scope.

### EXAMPLES

### Materials and Methods

### MD Simulations

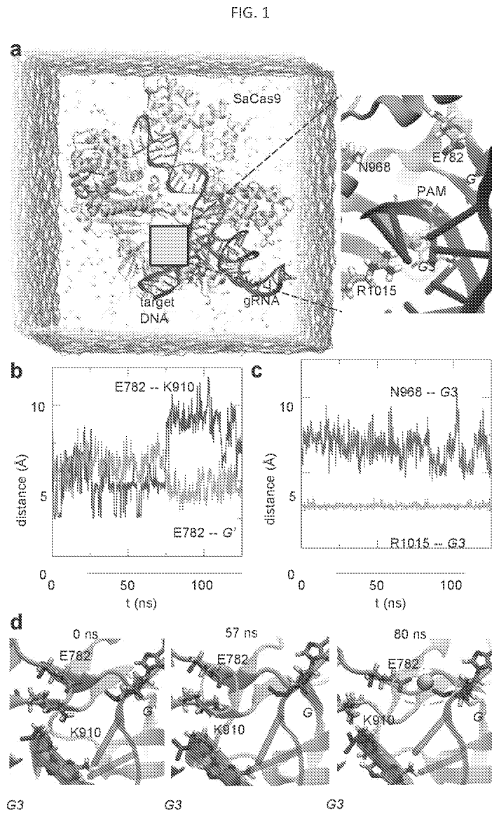

All-atom molecular dynamics (MD) simulations were performed on the SaCas9-sgRNA complex with or without bound DNA that is solvated in the 0.15 M NaCl electrolyte, as shown in FIG. 1A. Following previous protocols that were utilized for studying protein folding dynamics (Zhou et al., Proc. Natl. Acad. Sci. USA, 100: 13280-13285 (2003); Liu et al., Nature, 437: 159-162 (2005)), discovering molecular mechanism in protein-ligand binding (Want et al., Proc. Natl. Acad. Sci. USA, 109: 1937-1942 (2012); and Chipot, C. and A. Pohorille, Free Energy Calculations; Springer (2007)), investigating interactions at a bionano interface (Ge et al., Proc. Natl. Acad. Sci. U.S.A, 108: 16968-16973 (2011), Tu et al., Nature Nanotechnol., 8: 594-601 (2013); Luan et al., ACS Nano, 9: 663-669 (2015); Luan et al., ACS Nano, 11: 12615-12623 (2017)) and beyond, all MID simulations and FEP calculations were carried out with the software package NAMD2.11 (Phillips et al., J. Comp. Chem., 26: 1781-1802 (2005)). After solvating the complex (PDB ID: 5CZZ) in a cubic water box with each edge measuring about 126.3 Å, 249 Na⁺ and 175 Cl⁻ were added into the system, neutralizing the charge of the complex and setting the ion concentration to be 0.15 M, corresponding to the experimentally-validated active condition for SaCas9 (Ran et al., Nature, 520: 186-191 (2015)). Although Mg2+ or similar divalent metal ions are important for the DNA cleavage activity of RuvC and HNH domains in SaCas9, they are located at the nuclease domain active sites and do not bind to or affect the PAM recognition process, which is the focus of studies described herein. Hence, divalent ions were not included in this simulation. The final system, shown in FIG. 1A, contains 206,984 atoms and was minimized for 10 ps and further equilibrated for 10 ns in the NPT ensemble (P˜1 bar and T˜300 K), with atoms in backbones harmonically constrained (spring constant k=1 kcal/mol/Å2). After removing S9 constraints, the entire system was then equilibrated for additional 5 ns in the NPT ensemble, which was followed by production runs performed in the NVT ensemble.

The CHARMM force field (MacKerell et al., J Phys. Chem. B, 102: 3586-3616 (1998)) was applied for the protein, DNA, and RNA molecules; the TIP3P model (Jorgensen et al., J. Chem. Phys., 79: 926-935 (1983); Neria et al., J. Chem. Phys., 105: 1902-1921 (1996)) was chosen for water; the standard force field (Beglov, D. and B. Roux, J. Chem. Phys., 100: 9050-9063 (1994)) was used for ions. Periodic boundary conditions (PBC) were applied in all three dimensions. Long-range Coulomb interactions were calculated using particle-mesh Ewald (PMF) full electrostatics with the grid size about 1 Å in each dimension. The van derWaals (vdW) energies between atoms were calculated using a smooth (10-12 Å) cutoff. The temperature T was maintained at 300 K by applying the Langevin thermostat (Allen, M. P. and Tildesley, D. J., Computer Simulation of Liquids; Oxford University Press: New York (1987)) to all oxygen atoms in water and backbone atoms in simulated molecules. The pressure was kept constant at 1 bar using the Nose-Hoover method (Martinez, T. and K. Schulten, Neur Netw., 7: 507-522 (1994)). With the SETTLE algorithm (Miyamoto et al., J. Comp. Chem., 13: 952-962 (1992)) enabled to keep all bonds rigid, the simulation time-step was 2 fs for bonded and non-bonded (e.g., vdW, angle, and dihedral) interactions, and electric interactions were calculated every 4 fs, with multiple time-step algorithm (Tuckerman et al., The Journal of Chemical Physics, 97: 1990-2001 (1992); and Morrone et al., Journal of Chemical Theory and Computation, 6: 1798-1804 (2010)).

### Free Energy Perturbation Calculations

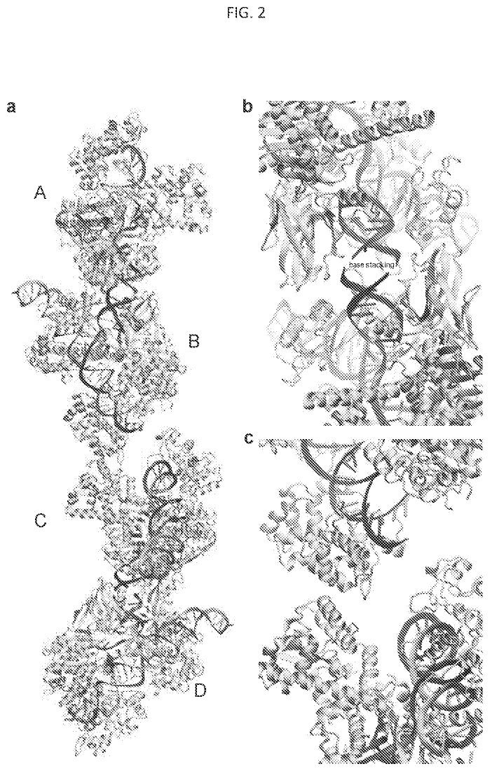

The free energy perturbation (FEP) method is described in Chipot, C.; Pohorille, A. Free energy calculations; Springer, 2007. Here, after obtaining the equilibrated bound and free states of complexes, this method was used to calculate the change of the binding free energy for each proposed mutation on SaCas9. FIG. 2A shows a thermal dynamical cycle used in the FEP method to calculate the free energy difference ΔΔG for the mutation R1015H: ΔG_{A} and ΔG_{B} are free energy changes for dsDNA.s binding to wild type SaCas9 and mutated SaCas9, respectively; ΔG₁ and ΔG₂ are free energy changes for annihilating R1015 and simultaneously creating H1015 in the bound (with dsDNA) and the free (without dsDNA) states, respectively.

For the R10151H mutation, the difference between dsDNA's binding free energies can be calculated by the following equation:

$$ΔΔ G = Δ {G}_{A} - Δ {G}_{B} = Δ {G}_{1} - Δ {G}_{2} , \qquad ( 1 )$$

Generally, direct calculations of ΔG_{A} and ΔG_{B} are challenging and can be circumvented by computing ΔG₁ and ΔG₂ instead (see equation 1 above). From the following ensemble average (Chipot, supra), ΔG₁ and ΔG₂ can be calculated theoretically using the following equation:

$$Δ G = - {k}_{B} Tln < exp ( \frac{{H}_{f} - {H}_{i}}{{k}_{B} T} ) > i \qquad ( 2 )$$

where k_{B} is the Boltzmann constant; T is temperature; and Hᵢ and H_{f} the Hamiltonians at the initial (i) and the final (f) stages respectively. For example, for the R1015H mutation, the initial state is the wild-type SaCas9 and the final state is the one with R1015 replaced by 1-I1015. Using the perturbation method, many intermediate stages (denoted by λ) whose Hamiltonian H(λ)=λH_{f}+(1−λ)Hᵢ ought to be inserted between initial and final states to improve the accuracy. In calculations of ΔG₁ and ΔG₂, λ changes from 0 to 1 in 18 perturbation windows with the soft-core potential enabled, yielding gradual annihilation and creation processes for R1015 and H1015, respectively.

### SaCas9 Experiments

Experimental assays were performed using constructs from original SaCas9 work with molecular cloning to introduce mutations or alterations that correspond to engineering design or computational simulation. The backbone vector used was the pX601-SaCas9 plasmid (available from Addgene) as previously described in Ran et al., Nature, 520: 186-19 (2015). Briefly, oligo primers (IDT DNA) were designed to amplify DNA fragments containing desirable mutations of the SaCas9 construct and used in a PCR reaction with template pX601 plasmid. The resulting PCR products were purified using a PCR purification kit (QIAGEN), subjected to further separation by agarose gel electrophoresis, and then purified again with a gel-extraction kit (QIAGEN) before normalization for downstream assembly. Final cloning of vectors was performed using the Gibson Assembly method and transformed into bacteria for isolating plasmids. All plasmids were verified by Sanger Sequencing (Genewiz) and stored for cell transfection experiments.

For measurement of SaCas9 activity in mammalian cells, human embryonic kidney 293FT cells (Thermo Fisher) were maintained in Dulbecco's modified Eagle's Medium (DMFM), supplemented with FBS and GlutaMAX (ThermoFisher), in incubators at 37° C. with 5% CO₂ supply. Around 24 hours prior to transfection, cells were seeded into 24-well plates (Corning) at a density of 2.5×10⁵ cells per well, and transfected at appropriate confluency using Lipofectamine 2000 (Thermo Fisher), according to the manufacturer's recommended protocol. A total of 600 ng DNA was used for each well of the 24-well plate. Cells were then incubated until ready to be harvested. Detection and quantification of genomic modification were done using the workflow similar to that described in, e.g., Cong et al., Science, 339: 819-823 (2013); and Nishimasu et al., Cell, 162: 1113-1126 (2015). Briefly, about 72 hours after transfection. genomic DNA from transfected cells was harvested using QuickExtract DNA Extraction Solution (Epicentre) with a step-wise incubation method, followed by InDel analyses using the SURVEYOR assay, as described previously (Cong et al., supra). The targeted genomic region was amplified using primers for the SURVEYOR assay with amplicon size between 500 to 900 bp for all targets. In the SURVEYOR assay, purified PCR product was re-annealed, subjected to SURVEYOR nuclease digestion, and then analyzed and quantified by polyacrylamide gel electrophoresis (Cong et al., supra). All experiments were done in triplicate to account for possible technical noise in the assay to obtain the error statistics.

### Example 1

This example demonstrates an all-atom molecular dynamics simulation of the SaCas9 complex.

An examination of the high-resolution SaCas9 complex structure (Nishimasu et supra) was performed (although crystal contacts may affect local structure; see FIG. 2). To establish dynamic details of the SaCas9 complex in its native state, a molecular dynamics (MD) method was used to model the complex under physiological conditions. MD simulations, complimentary to experimental studies, have proven to be effective in understanding protein-DNA interactions Palermo et al., Proceedings of the National Academy of Sciences, 114: 7260-7265 (2017); and Cong et al., Nat. Commun., 3: 968 (2012)). All-atom MD simulations were performed to characterize the molecular mechanism of the binding of a DNA target with an SaCas9-sgRNA complex (see FIG. 1A).

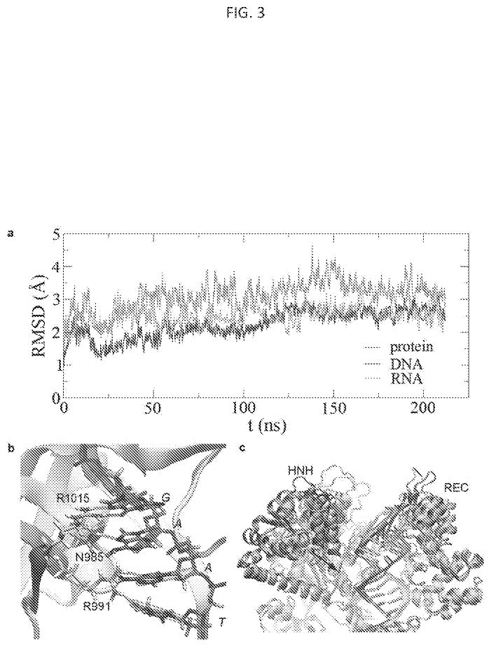

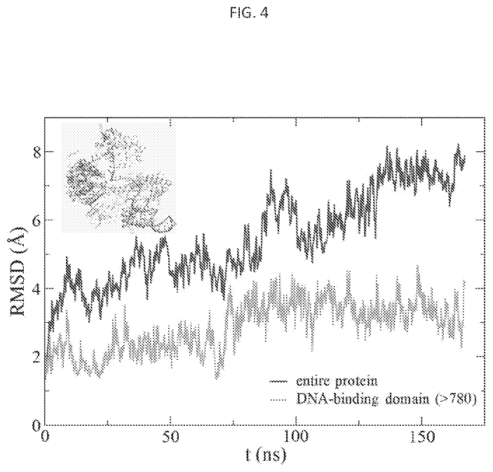

In the MD analysis, after equilibration in the bound state with target DNA substrate (FIG. 3A), the secondary structures of RNA and DNA molecules were stable as the averages of saturated root-mean-square-deviations (RMSDs) calculated against the crystal structures were both around 2.5 Å. Following a similar protocol, the equilibrated complex without bound DNA was independently obtained. From a global view, the saturated RMSD of backbone atoms was only about 3.2 Å in the SaCas9 complex with substrate DNA (FIG. 3A), while the HNH domain of the nuclease (NUC) lobe (the HNH domain cleaves the DNA strand complementary to the guide RNA sequence), without being blocked by the recognition (REC) lobe from a neighboring protein in the crystal environment (FIG. 2C), moved a distance of 7.6 Å toward the cleavage site on the target DNA strand, which exactly recapitulated the physiological process (FIG. 3C). Similarly, the REC lobe as well as the end fragment of DNA-RNA heteroduplex moved closer to the NUC lobe (FIG. 3C), On the other hand, for SaCas9 without the bound DNA (FIG. 4), RMSDs increased and saturated at 7.5 Å, indicating a larger conformational change in relevant domains. Nonetheless, RMSDs for the DNA binding region (where proposed mutations lie) remained small (about 3.5 Å) (FIG. 4). These observations are all in accordance with biochemical and biophysical analysis in previous reports (Sternberg et al., Nature, 527: 110 (2015); Jiang et al., Science, 351: 867-871 (2016); Dagdas et al., Science Advances, 3: eaao0027 (2017); and Chen et al., Nature, 550: 407 (2017)). With these equilibrated structures from both bound and free states relating well to experimental findings, the molecular basis of SaCas9 PAM recognition was then investigated, and free-energy perturbation (FEP) calculations were performed on mutations of novel SaCas9 variants (i.e., in silico mutagenesis studies).

As enlarged and highlighted in FIG. 1A, the binding site in the PAM-interacting (PI) domain contains all three residues from KKR SaCas9, namely E782, N968, R1015, along with the G in NNGRRT, whose specificity was altered (Kleinstiver et al., Nature Biotech., 33: 1293-1298 (2015)). Hereafter, G3 was used to denote the third position of PAM (the base changed in KKH SaCas9 PAM) and G0 was used to denote the first nucleotide at the PAM proximal end on target DNA strand. From MD results, all key PAM recognizing residue interactions were observed in crystal structure: R1015 coordinates the G3; N985 coordinates the A at the fourth position; and 8991 dynamically coordinates both the A and the T at the fifth and the sixth positions, respectively (see FIG. 3B). The role of R991 in coordinating A (or R generally) at the fifth position could not be derived from the (static) crystal structure where none of the residues are in contact with the A.

At a close-in view for finer details, several key residues located near the PI domain were observed to adjust side-chain positions compared with those in the crystal. This could be due to an environment that bears closer resemblance to the in vivo active state. By calculating distances of residue pairs (defined as the shortest distance between non-hydrogen atoms), it was found that residue E782 could be close to either K910 or the G0, signaling its possible direct involvement in PAM interaction (see FIG. 1B). On the other hand, the flexible N968 was near the G3, but it was not close enough to form a direct contact (see FIG. 1C). Notably, K910 formed dynamic coordinations that were not shown in the crystal structure. FIG. 1D shows the conformation of K910 (in crystal environment at 0 ns) that was sandwiched by negatively charged E782 and G3. However, the positively-charged amine group (NH3+) in K910 did not form a salt bridge with either the carboxyl group (COO⁻) of E782 or the phosphate group (PO₄⁻¹) of G3. During MD simulation, K910 moved toward E782 and formed a salt bridge with E782 after 57 ns (FIG. 1D). This salt-bridge was broken later after a Na+ diffused into this region. FIG. 1D shows that, at 80 ns, K910 formed a new salt bridge with G3, while at the same time E782 bound this Na+, further coordinating the phosphate group of G0 in target DNA strand to stabilize dsDNA binding. These coordinations were absent in substrate-free state and further demonstrates the critical role of dynamic conformational transitions for strong PAM recognition of SaCas9.

### Example 2

This example describes the use of free-energy perturbation and experimental assays to probe Cas9 PAM recognition.

Completing system equilibrations and MD analysis revealed previously under-appreciated dynamics of PAM recognition and addressed one of the fundamental challenges in modeling genome editing tools, which is how to inform the contribution of protein residues to target recognition in a quantitative manner. To this end, a combined process was utilized where structural insight guided computational analysis, followed by targeted gene editing experimentation that justified further computational mapping of Cas9 variant activity, where in-silico prediction could correlate with experimental Cas9 editing efficiency.

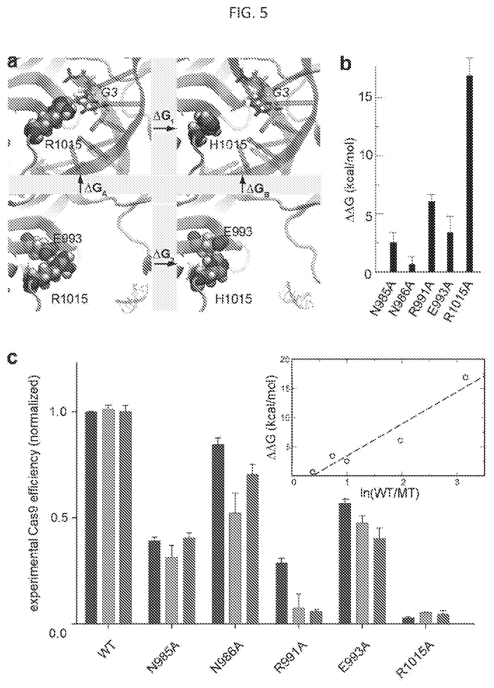

First, the contribution of PI domain residues to SaCas9 PAM recognition was quantified with free energy perturbation (FEP) calculations (FIG. 5A). Alanine scan analysis of the residues directly in vicinity of the PAM sequence was performed. FIG. 5B shows that mutations R991A and R1015A significantly reduced the binding free energies, while N986A (due to the small value of ΔΔG) was much less important. Mutations N985A and E993A also resulted in about 2-4 kcal/mol increases in ΔΔG, which could destabilize the PAM binding. Experimentally, corresponding SaCas9 mutants bearing targeted alanine mutations were generated and expressed with guide RNAs (gRNAs) to quantitatively assess Cas9 activity, measured as its cleavage efficiency across three different genomic targets. The efficiency against wild-type Cas9 was normalized so that reduction of activity after introducing alanine mutations would indicate the importance of the particular residue tested (FIG. 5C). Next, to examine the correlation between computational and experimental data, linear fitting of DDG was performed from each alanine mutation versus the transformed activity of experimental counterpart (calculated by taking the natural log of mutant SaCas9 efficiency over wild-type control), as plotted in FIG. 5C (inset). Measured biological activity matched the FEP calculation well, as indicated by goodness of fitting reaching 0.92 (FIG. 5C inset). Overall, these results revealed strong predictability of this COmbined Molecular dynamics and Experimental Target (“COMET”) validation approach, and possible non-linear factors were noted that may affect the computational-experimental translation, such as endogenous genome context.

### Example 3

This example describes an analysis of the KKH SaCas9 variant to reveal the molecular mechanism of its expanded PAM.

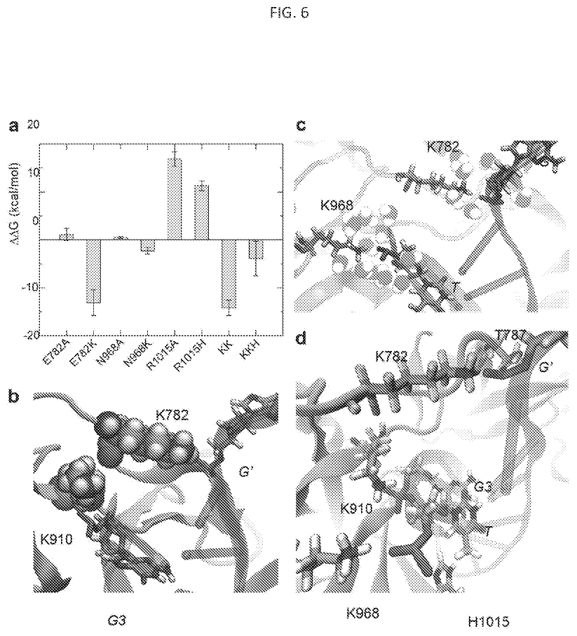

The KKH mutant of SaCas9 involves three substitutions: E782K, N968K and R1015H (Kleinstiver et al., Nature Biotech., 33: 1293-1298 (2015)). The thermodynamic cycle for R1015H is illustrated in FIG. 5A. In the bound state, R1015 binds the G3 with two hydrogen bonds, responsible for the PAM specificity NNGRRT. This interaction was further stabilized by a salt bridge between E993 and R1015 that can significantly reduce the conformational fluctuation of R1015. The same salt bridge is also present in the free state of SaCas9 as shown in FIG. 5A. After the R1015H mutation, in the bound state H1015 moved away from G3, thereby releasing the specificity on G3 in the NNGRRT PAM. However, such mutation (denoted by ΔG1 in FIG. 5A) significantly reduced the binding free energy (or binding affinity). Compared with the same mutation process in the free state (denoted by ΔG2 in FIG. 5A), the net change of the binding free energy was +11.3 kcal/mol (FIG. 3A). This was a significant reduction in binding affinity, which was even more unfavorable when compared with the ΔΔG for the R1015A mutation (about 16.9 kcal/mol, see FIG. 5B). Thus, despite reducing the PAM specificity, the binding between the PI domain of SaCas9 and the PAM region of dsDNA was destabilized by R1015H. To compensate for this and stabilize protein-DNA binding, extra mutations (E782K and N968K) were introduced in previous work (Kleinstiver et al., supra). As shown in FIG. 1D, the E782K mutation was expected to have profound changes on local coordinations: 1) the N³⁺ group of K782 binds to the phosphate group of G0 in the target DNA strand directly; 2) K910 repelled by K782 binds to G3 more stably. Indeed, in the final stage of the FEP calculation, K910 and K782 were bound to G3 and G0 in two complimentary DNA strands, respectively, via the salt-bridge formed by the amine and the phosphate groups (FIG. 6B), which can significantly increase DNA-protein binding free energy, Consistently, the calculated ΔΔG for the E782K mutation was −13.1 kcal/mol, which was much more favorable than the E782A mutation, which destabilized the local coordination (E782-Na+-G0, FIG. 1D) with a calculated. DDG of about 1.1 kcal/mol (FIG. 6A). Furthermore, the N968K mutation was suggested to enhance substrate binding (Kleinstiver et al., supra). Consistent with the experimental result, FEP calculation revealed that the ΔΔG for this residue change was about −2.3 kcal/mol (FIG. 6A). In the end of FEP analysis for the bound state, K968 could move close to G3 in the PAM sequence because of electrostatic attraction between the amine group in K968 and the phosphate group in G3. K910 could momentarily bind to G3 as well (FIG. 1D). Therefore, the smaller free energy reduction for the N968K mutation as compared to E782K was the result of the temporary electrostatic repulsion between K910 and K968, i.e., a weaker binding between K968 and G3 of the PAM.

Mutating N968 to alanine had a negligible effect on protein-DNA binding (ΔΔG=0.5 kcal/mol), indicating its relative neutrality on PAM recognition in wild-type SaCas9 which corroborates earlier results from MD simulation (FIG. 1C). The double mutations E782K and N968K (KK) yielded even stronger protein-DNA binding, enhancing the binding free energy by 14.2 kcal/mol (FIG. 6A). If acting by a simple additive manner, the KK double mutation was expected to at least increase the binding free energy by 15.3 kcal/mol (via addition of two −ΔΔG values). As a consequence of the E782K mutation, K910 can stably bind to G3 in the PAM sequence, However, K968 can also competitively interact with the same G3. Thus, the change of −ΔΔG for the KK mutation is less than the simple addition of two independent ones, indicating a complex interplay among these mutated residues. Finally, simultaneous triple mutations E782K, N968K, and R1015H (KKH) yielded ΔΔG of −3.9 kcal/mol, a net gain in binding free energy (FIG. 6A). As expected, when the specific binding between R1015 and G3 was released, the PAM region on the non-target DNA strand was allowed larger conformational fluctuation as indicated by the entropy calculation. Briefly, before (1=0) and after (1=1) the FEP calculation of the KKH mutation, the simulation system was run for an extra 2 ns to sample conformations of the triplet TGA in the PAM sequence TTGAAT. It was expected that after the R1015H mutation the nucleotide G in the PAM sequence would be less coordinated by the PI domain and may have a larger fluctuation. The Schlitter method (Schlitter, J., Chemical Physics Letters, 215: 617-621 (1993)) to calculate the conformational entropies before and after the KKH mutation. Results show that conformation entropies of the triplet changed from 1400 J/(mol·K) to 1341 J/(mol·K) due to the KKH mutation. Consequently, the FEP computation shows that K968 can bind to the phosphate group of T (one nucleotide ahead of the G3 in TTGAAT PAM) and K910 can bind to G3, which reduces the electrostatic repulsion between K968 and K910, improving the affinity of protein-DNA binding (FIG. 6C).

The salt bridge formed by K968 and the T are well exposed to water, with 12 water molecules within 4 Å of the salt bridge (FIG. 6C). However, the salt bridge formed by K782 and the G0 are considerably buried within the complex, with only 6 water molecules within 4 Å of the salt bridge. Thus, due to the different dielectric environments, the binding free energy enhancement from the K968-T salt-bridge can be much smaller than that for the K782-G0 salt bridge (Zhou, R., Proc. Natl. Acad. Sci. USA, 100: 13280-13285 (2003)). Thus, based on the FEP calculation, the KKH mutations were able to only modestly enhance protein-DNA binding (FIG. 6A), taking into account the error of the analysis. The molecular mechanism of KKH mutations is summarized in FIG. 3D, as E782K and N968K compensate for the free energy loss by the R1015H mutation that removes the restriction to G3 in the PAM, leading to expanded targeting range of KKH SaCas9 without compromising its energetic property.

In addition to the energy calculation, the simulation revealed that all other coordinations between wild-type SaCas9 and the bound DNA were preserved. For example, the phosphate locker T787 forms a hydrogen bond with the G0 (FIG. 6D) and R991 coordinates AT in the TTGAAT PAM, which are both key residues involved in target DNA binding.

### Example 4

This example describes COMET-based engineering of SaCas9 variants to expand PAM range.

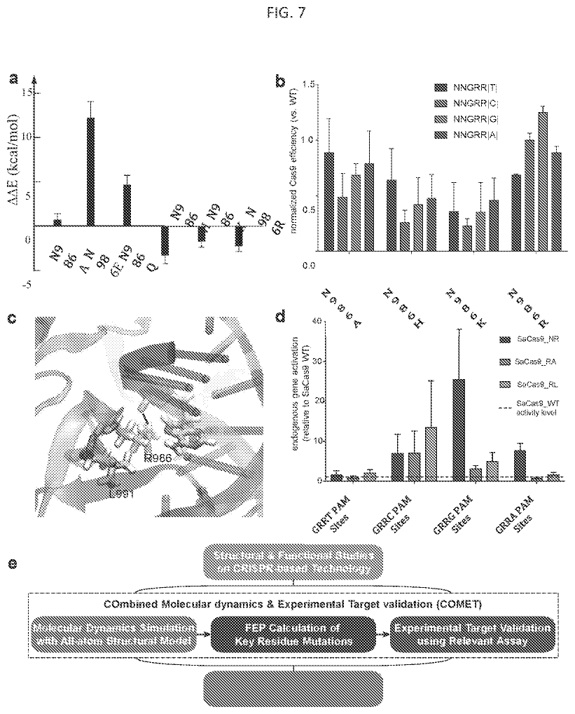

The analysis of KKH SaCas9 described above and its consistency with previous experiments led to the expansion of the COMET approach for rational exploration of novel SaCas9 designs to alter PAM specificity. To this end, the remaining non-wobbling position of SaCas9 PAM was targeted, i.e., the last (sixth) T base of NNGRRT, a prime constraint in gene editing applications. From structural information and the above-described MD simulation, N986 serves as the key residue for coordinating this PAM position. Hence, as a first step, a screening of various mutations was performed on N986 to change it to alternative amino acids (mostly charged for maintaining protein-DNA interaction) using the COMET workflow, and yielded a set of FEP calculations to guide downstream experiments (FIG. 7A). Based on the free energy results, the most promising candidates were N986H/K/R mutants. The unfavorable energy prediction for N986A, N986E, and N986Q mutants guided experimental efforts so that these variants could be excluded from experimental tests. Here, the COMET workflow spared significant time and cost given that, for defining Cas9 PAM specificity, each individual mutant would have to be tested against complete sets of editing sites spanning four different bases at the target position, i.e. NNGRRT=C=G=A. The targeted experiment on SaCas9 N986H/K/R variants revealed that their PAM recognition profiles were indeed modified to various degree, with SaCas9 N986R as the single most notable candidate (FIG. 7B). Compared with the wild type, SaCas9 N986R moderately preferred the non-natural PAM NNGRRG, with decreased activity against NNGRRT while mostly maintaining the PAM recognition activity of other bases on the sixth PAM position. As expected, a single mutation could affect, but not sufficiently create, a powerful new variant, demanding combination effects from additional mutations to probed with another iteration of COMET.

To guide the selection of additional target residues for combinatorial mutagenesis, the new MD simulation modeling was performed on the top-ranked SaCas9 N986R variant to probe its PAM recognition process (FIG. 7C). From the molecular details of residue coordination, it was hypothesized that R991, in close proximity to N986R, would potentially interact with N986R in a negative fashion. Hence, following computationally screening of possible mutations on R991, the R991A/L/K variant was combined with N986R to further enhance its non-T PAM recognition. With this lead from the COMET workflow, DNA targeting assays were applied to test base preference of the last PAM position of these combinatorial SaCas9 variants, again compared with wild type reference.

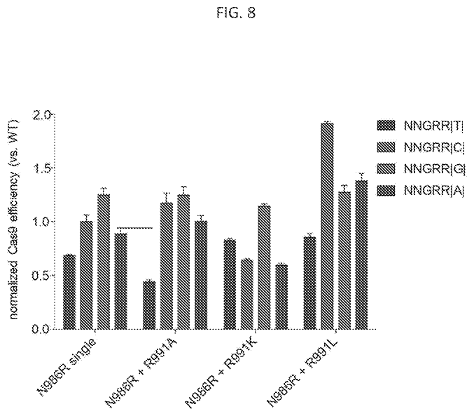

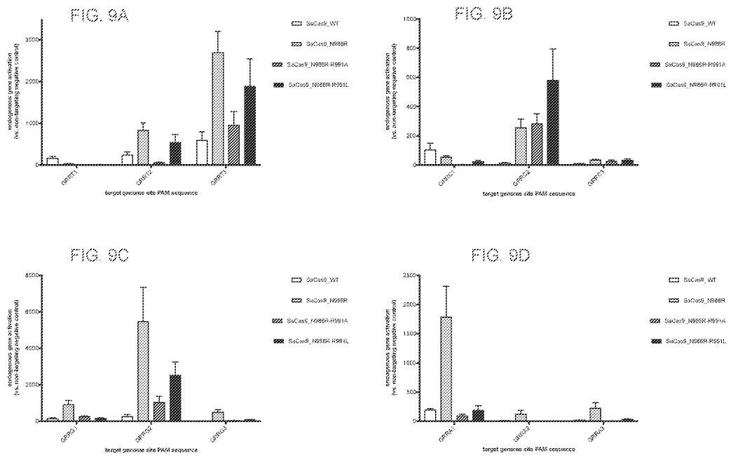

The results yielded another candidate non-T PAM SaCas9 variant, SaCas9 N986R R991L, which showed significantly enhanced recognition of NNGRRC and NNGRRG, as well as moderately improved NNGRRA PAM binding activity across different targets when applied to target endogenous genome sequences (see FIG. 8). The activity of both variants, when compared to the original SaCas9, for the fiat time allows for efficient targeting of new PAM sequences that were previously inaccessible to this small Cas9, with potentially triple or quadruple expansion of SaCas9 range of action, as demonstrated in human cells (FIG. 7D, FIG. 9). These encouraging results verified on multiple targets within mammalian cell contexts led to terming these new variants as SaCa.s9-NR (for SaCas9 N986R) and SaCas9-RL (for SaCas9 N986R+R991L). These SaCas9 variants serve as a promising component in the family of Cas9 tools for targeting disease-relevant loci where the last position in SaCas9 natural PAM prevents optimal design of editing strategy. The expansion may enhance the range of available small Cas9 tools, particularly given the ability to combine SaCas9-NR and SaCas9-RL with other powerful Cas9-based tools for enhancement (Slaytnaker et al., Science, 351: 84 (2016)).

These results demonstrated the capacity of COMET to engineer novel Cas9 proteins with modified properties.

### Example 5

This example describes additional SaCas9 mutants that provide stronger interaction with PAM duplex and have higher activity.

New SaCas9 variants were generated using molecular cloning to introduce mutations or alterations that correspond to engineering design or computational simulation. The backbone vector used was the pX601-SaCas9 plasmid (available from Addgene) as previously described. Briefly, oligo primers (IDT DNA) were designed to amplify DNA fragments containing desirable mutations of the SaCas9 construct and used in a PCR reaction with template pX601 plasmid. The resulting PCR products were purified using a PCR purification kit (QIAGEN), subjected to further separation by agarose gel electrophoresis, and purified again with gel-extraction kit (QIAGEN) before normalization for downstream assembly. Final cloning of vectors was performed using the Gibson Assembly method and transformed into bacteria for isolating plasmids. All plasmids were verified by Sanger Sequencing (Genewiz) and stored for cell transfection experiments.

For measurement of SaCas9 activity in mammalian cells, human embryonic kidney 293T cells were maintained in Dulbecco's modified Eagle's Medium (DMEM), supplemented with FBS and GlutaMAX (Thermo Fisher), in incubators at 37° C. with 5% CO₂ supply. Around 24 hours prior to transfection, cells were seeded into 24-well plates (Corning) and transfected at appropriate confluency using Lipofectamine 2000 (Thermo Fisher), according to the manufacturer's recommended protocol. A total of 600 ng to 800 ng DNA was used for each well of the 24-well plate. Cells were then incubated until ready to be harvested.

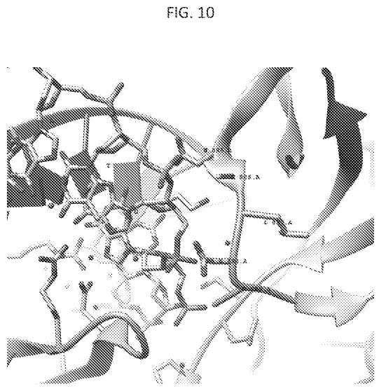

With additional structural and computational analysis, additional amino acid residues were identified within the SaCas9 protein as potential candidates for improving the PAM recognition activity of SaCas9. It was concluded that these may be synergistic to our existing variants. The amino acid residues experimentally tested were: N885; K886; L887; N888; A889. As shown in FIG. 10, these residues do have a relative short distance with the PAM duplex located within the target DNA site. Hence, new SaCas9 variants were generated bearing additional amino acid mutations in combination with the two top variants: (1) SaCas9-N986R (SaCas9-NR), labeled in FIG. 10 as 9868; (2) SaCas9-N986R/R991L (SaCas9-RL), labeled in FIG. 10 as 986R/991L. The original 986R and 986R/991L variants were included in the tests as reference.

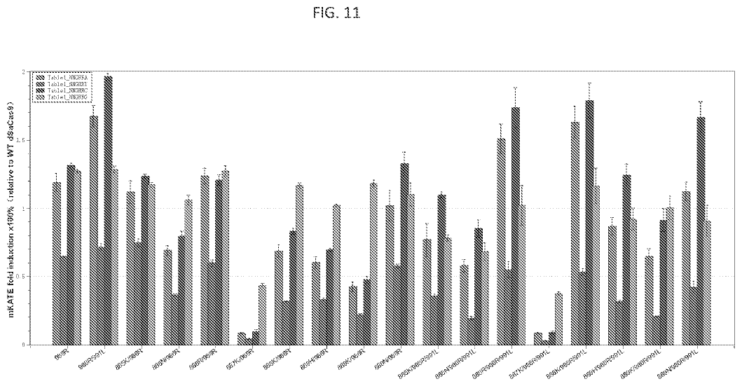

In these new variants, original amino acid residues were mutated to highly charged residues to provide stronger, more favorable interaction with the PAM duplex for higher binding activity, as shown in FIG. 11.

### Example 6

This example describes additional structural modeling and experiments indicating that mutations on E782, N968 have combinatorial enhancement for existing SaCas9 variants.

Previous rounds of mutation led to additional analysis of the SaCas9 protein and the proposal of additional residues to mutate and test. These new amino acid residues, namely E782 and N968, are located in structural distinctive regions from the set of residues mutated in Example 5 (N885; K886; L887; N888; A889), which focused on binding to PAM duplex of target DNA site. Instead, the E782 and N968 residues in this analysis could potentially enhance the general binding of the SaCas9 with its target DNA but not necessarily confined to the PAM duplex. Hence, the combinations of these residue mutations with the other variants described herein may create “v2.0” of the SaCas9-NR and SaCas9-RL variants that further strengthen their ability to bind DNA targets with non-natural PAM sequence, with higher gene-editing activity. Of note, these two residues were part of a design previously shown to bind a different PAM sequence (Kleinstiver et al., supra).

Mutants bearing either E782K or N968K in combination with the SaCas9-NR and SaCas9-RL mutations were able to enhance existing variants. The E782K¹N986R, N968K/N986R, E782K/N986R/R991L, N968K/N986R/R991L were the top variants that have higher efficiency on the non-natural PAM sequence NNGRR[A/C/G]. These variants constitute a series of v2.0 SaCas9 variants, which are set forth in Table 1.

TABLE 1

| | | Cas9 Activity on different PAM sequences (normalized to | | | |
|---|---|---|---|---|---|
| | | WT average over all target DNA sites) | | | |
| | | Target Site | Target Site | Target Site | Target Site |
| SaCas9 | | PAM = | PAM = | PAM = | PAM = |
| Variant Name | Mutation detail | NNGRRT | NNGRRC | NNGRRG | NNGRRA |
| SaCas9 Wild | No mutation | 1.000 | 1.000 | 1.000 | 1.000 |
| Type (WT) | | | | | |
| SaCas9-NA | N986A | 0.888 | 0.488 | 0.687 | 0.791 |
| SaCas9-NR | N986R | 0.685 | 1.001 | 1.250 | 0.888 |
| SaCas9-NK | N986K | 0.360 | 0.229 | 0.350 | 0.459 |
| SaCas9-NH | N986H | 0.643 | 0.261 | 0.420 | 0.478 |
| SaCas9-RA | N986R + R991A | 0.444 | 1.174 | 1.248 | 1.002 |
| SaCas9-RK | N986R + R991K | 0.827 | 0.642 | 1.144 | 0.597 |
| SaCas9-RL | N986R + R991L | 0.854 | 1.909 | 1.273 | 1.380 |
| SaCas9-RAR | N986R + R991A + T1019R | 0.437 | 0.437 | 0.946 | 0.421 |
| SaCas9-RAK | N986R + R991A + T1019K | 0.226 | 0.226 | 0.647 | 0.256 |
| SaCas9-RAH | N986R + R991A + T1019H | 0.365 | 0.365 | 1.257 | 0.508 |
| SaCas9-RKR | N986R + R991K + T1019R | 0.289 | 0.289 | 0.839 | 0.182 |
| SaCas9-RKK | N986R + R991K + T1019K | 0.183 | 0.183 | 0.518 | 0.149 |
| SaCas9-RKH | N986R + R991K + T1019H | 0.639 | 0.639 | 1.231 | 0.462 |
| SaCas9-RLR | N986R + R991L + T1019R | 0.671 | 0.671 | 1.277 | 0.739 |
| SaCas9-RLK | N986R + R991L + T1019K | 0.730 | 0.730 | 1.107 | 0.847 |
| SaCas9-RLH | N986R + R991L + T1019H | 0.545 | 0.545 | 1.211 | 0.798 |
| SaCas9-RCR | N986R + R991C + T1019R | 0.447 | 0.447 | 1.310 | 0.529 |
| SaCas9-RCK | N986R + R991C + T1019K | 0.334 | 0.334 | 1.120 | 0.449 |
| SaCas9-RCH | N986R + R991C + T1019H | 0.325 | 0.325 | 1.097 | 0.419 |
| SaCas9-RVR | N986R + R991V + T1019R | 0.421 | 0.421 | 0.769 | 0.473 |
| SaCas9-RVK | N986R + R991V + T1019K | 0.478 | 0.478 | 0.896 | 0.482 |
| SaCas9-RVH | N986R + R991V + T1019H | 0.270 | 0.270 | 0.525 | 0.400 |
| SaCas9- | N885K + N986R | 0.746 | 1.233 | 1.173 | 1.118 |
| NewDouble-M1 | | | | | |
| SaCas9- | K886N + N986R | 0.364 | 0.795 | 1.060 | 0.690 |
| NewDouble-M2 | | | | | |
| SaCas9- | K886R + N986R | 0.598 | 1.207 | 1.273 | 1.237 |
| NewDouble-M3 | | | | | |
| SaCas9- | L887K + N986R | 0.042 | 0.095 | 0.435 | 0.085 |
| NewDouble-M4 | | | | | |
| SaCas9- | N888K + N986R | 0.316 | 0.830 | 1.165 | 0.686 |
| NewDouble-M5 | | | | | |
| SaCas9- | A889H + N986R | 0.331 | 0.695 | 1.022 | 0.600 |
| NewDouble-M6 | | | | | |
| SaCas9- | A889K + N986R | 0.218 | 0.475 | 1.181 | 0.425 |
| NewDouble-M7 | | | | | |
| SaCas9- | A889N + N986R | 0.576 | 1.325 | 1.102 | 1.019 |
| NewDouble-M8 | | | | | |
| SaCas9-NewTriple-M1 | N885K + N986R + R991L | 0.355 | 1.097 | 0.780 | 0.768 |
| SaCas9-NewTriple-M2 | K886N + N986R + R991L | 0.194 | 0.851 | 0.682 | 0.579 |
| SaCas9-NewTriple-M3 | K886R + N986R + R991L | 0.550 | 1.736 | 1.021 | 1.509 |
| SaCas9-NewTriple-M4 | L887K + N986R + R991L | 0.028 | 0.089 | 0.374 | 0.085 |
| SaCas9-NewTriple-M5 | N888K + N986R + R991L | 0.533 | 1.787 | 1.164 | 1.629 |
| SaCas9-NewTriple-M6 | A889H + N986R + R991L | 0.316 | 1.242 | 0.920 | 0.868 |
| SaCas9- | A889K + N986R + R991L | 0.206 | 0.912 | 1.004 | 0.645 |
| NewTrip1e-M7 | | | | | |
| SaCas9- | A889N + N986R + R991L | 0.418 | 1.666 | 0.908 | 1.123 |
| NewTriple-M8 | | | | | |
| SaCas9_NR_V2.1 | E782K + N986R | 1.014 | 1.760 | 1.212 | 1.873 |
| SaCas9_NR_V2.2 | N968K + N986R | 1.194 | 2.158 | 1.391 | 2.298 |
| SaCas9_NR_V2.3 | E782K + N968K + N986R | 0.457 | 0.789 | 1.129 | 0.961 |
| SaCas9_NR_V2.4 | E782K + N968R + 1015H | 0.027 | 0.066 | 0.415 | 0.072 |
| SaCas9_NR_V2.5 | N968K + N986R + 1015H | 0.027 | 0.057 | 0.400 | 0.069 |
| SaCas9_NR_V2.6 | E782K + N968K + N986R + 1015H | 0.048 | 0.133 | 0.521 | 0.123 |
| SaCas9_RL_V2.1 | E782K + N986R + R991L | 1.217 | 2.159 | 1.315 | 2.454 |
| SaCas9_RL_V2.2 | N968K + N986R + R991L | 1.255 | 2.498 | 1.455 | 2.768 |
| SaCas9_RL_V2.3 | E782K + N968K + N986R + R991L | 0.792 | 1.373 | 1.023 | 1.586 |
| SaCas9_RL_V2.4 | E782K + N986R + R991L + 1015H | 0.026 | 0.079 | 0.382 | 0.067 |
| SaCas9_RL_V2.5 | N968K + N986R + R991L + 1015H | 0.024 | 0.052 | 0.404 | 0.057 |
| SaCas9_RL_V2.6 | E782K + N968K + N986R + | 0.057 | 0.245 | 0.519 | 0.154 |
| | R991L + 1015H | | | | |

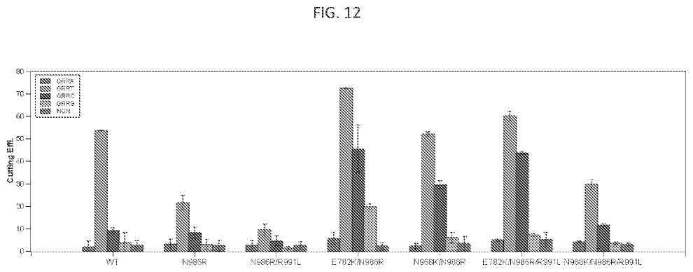

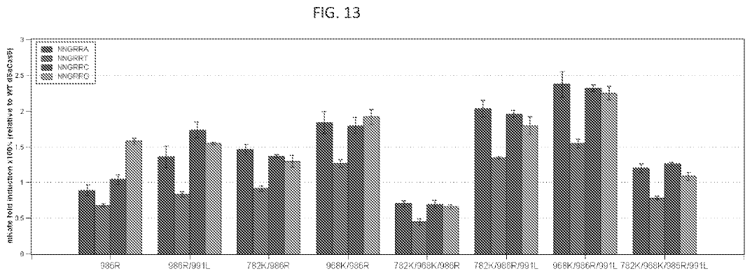

The difference (decoupling) between binding and cutting was revealed by the tests measuring binding (FIG. 12) and genome cleavage/editing (FIG. 13). For example, SaCas9-E782K/N986R did not have high binding activity, but demonstrated high genome cutting activity. On the other hand, SaCas9-N968K/N986R/R991L was a good target binder, but was not as efficient at cutting genomic DNA sites.

The additional “v2.0” SaCas9s described herein can be used for binding-based gene activation/repression or cutting-based gene editing. Specific SaCas9 variants may be selected based on the desired application for optimal results.

SEP ID NO: 1
KRNYILGEDIGITSVGYGIIDYETRDVIDAGYRLFK
EANVENNEGRRSKRGARRLKRRRRHRIQRVKKLLF
DYNLLTDHSELSGINPYEARVKGLSQKLSEEEFSA
ALLHLAKRRGVHNVNEVEEDTGNELSTKEQISRNS
KALEEKYVAELQLERLKKDGEVRGSINRFKTSDYV
KEAKQLLKVQKAYHQLDQSFIDTYIDLLETRRTYY
EGPGEGSPFGWKDIKEWYEMLMGFICTYFPEELRS
VKYAYNADLYNALNDLNNLVITRDENEKLEYYEKF
QTIENVFKQKKKPTLKQIAKEILVNEEDIKGYRVT
STGKPEFTNLKVYHDIKDITARKEIIENAELLDQF
AKILTIYQSSEDIQEELTNLNSELTQEEIEQISNL
KGYTGTHNLSLKAINLILDELWHTNDNQIAIFNRL
KLVPKKVDLSQQKEIPTTLVDDFILSPVVKRSFIQ
SIKVINAIIKKYGLPNDIIIELAREKNSKDAQKMI
NEMQKRNRQTNERIEEIIRTTGKENAKYLIEKIKL
HDMQEGKCLYSLEAIPLEDLLNNPFNYEVDHIIPR
SVSFDNSFNNKVLVKQEENSKKGNRTPFQYLSSSD
SKISYETFKKHILNLAKGKGRISKTKKEYLLEERD
INRFSVQKDFINRNLVDTRYATRGLMNLLRSYFRV
NNLDVKVKSINGGFTSFLRRKWKFKKERNKGYKHH
AEDALIIANADFIFKEWKKLDKAKKVMENQMFEEK
QAESMPEIETEQEYKEIFITPHQIKHIKDFKDYKY
SHRVDKKPNRELINDTLYSTRKDDKGNTLIVNNLN
GLYDKDNDKLKKLINKSPEKLLMYHHDPQTYQKLK
LIMEQYGDEKNPLYKYYEETGNYLTKYSKKDNGPV
IKKIKYYGNKLNAHLDITDDYPNSRNKVVKLSLKP
YRFDVYLDNGVYKFVTVKNLDVIKKENYYEVNSKC
YEEAKKLKKISNQAEFIASFYNNDLIKINGELYRV
IGVNNDLLNRIEVNMIDITYREYLENMNDKRPPRI
IKTIASKTQSIKKYSTDILGNLYEVKSKKHPQIIK
KG

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the intention unless otherwise indicated herein or otherwise clearly contradicted by context.

1. A variant Staphylococcus aureus Cas9(SaCas9) protein comprising the amino acid sequence of SEQ ID NO: 1, wherein one or more residues of E782, N968, N⁻986, and 8991 are substituted with a different amino acid.

2. The variant SaCas9 protein of claim 1, wherein the amino acid residue N986 of SEQ ID NO: 1 is substituted with a different amino acid.

3. The variant SaCas9 protein of claim 1 or claim 2, wherein the amino acid substitution is selected from N986A, N986R, N986K, and N986H.

4. The variant SaCas9 protein of claim 1, wherein the amino acid residue R991 of SEQ ID NO: 1 is substituted with a different amino acid.

5. The variant SaCas9 protein of claim 1 or claim 4, wherein the amino acid substitution is selected from R991A, R991K, R991L, R991C, and R991V.

6. The variant SaCas9 protein of any one of claims 1-5, wherein both of amino acid residues N986 and 8991 of SEQ ID NO: 1 are substituted with a different amino acid.

7. The variant SaCas9 protein of any one of claims 1-6, which further comprises an amino acid substitution of one or more residues of SEQ ID NO: 1 selected from E782, N885, K886, L887, N888, A889, N968, R1015, and T1019.

8. The variant SaCas9 protein of claim 7, further comprising one or more of the following amino acid substitutions: E782K, N885K, K886N, K886R, L887K, N888K, A889H, A889K, A889N, N968K, R1015H, T1019R, T1019K, and T1019H.

9. The variant SaCas9 protein of claim 8, which comprises the amino acid sequence of SEQ ID NO: 1 and two or more amino acid substitutions selected from:
(a) N986R and R991A;
(b) N986R and R991K;
(c) N986R and R991L;
(d) N986R, R991A, and T1019R;
(e) N986R, R991A, and T1019K;
(f) N986R, R991A, and T1019H;
(g) N986R, R991K, and T1019R;
(h) N986R, R991K, and T1019K;
(i) N986R, R991K, and T1019H;
(j) N986R, R991L, and T1019R;
(k) N986R, R991L, and T1019K;
(l) N986R, R991L, and T1019H;
(m) N986R, R991C, and T1019R;
(n) N986R, R991C, and T1019K;
(o) N986R, R991C, and T1019H;
(p) N986R, R991V, and T1019R;
(q) N986R, R991V, and T1019K;
(r) N986R, R991V, and T1019H;
(s) N885K and N986R;
(t) K886N and N986R;
(u) K886R and N986R;
(v) L887K and N986R;
(w) N888K and N986R;
(x) A889H and N986R;
(y) A889K and N986R;
(z) A889N and N986R;
(aa) N885K, N986R, and R991L;
(bb) K886N, N986R, and R991L;
(cc) K886R, N986R, and R991L;
(dd) L887K, N986R, and R991L;
(ee) N888K, N986R, and R991L;
(if) A889H, N986R, and R991L;
(gg) A889K, N986R, and R991L;
(hh) A889N, N986R, and R991L;
(ii) E782K and N986R;
(jj) N968K and N986R;
(kk) E782K, N968K, and N986R;
(ll) E782K, N986R, and R1015H;
(mm) N968K, N986R, and R1015H;
(nn) E782K, N968K, N986R, and R1015H;
(oo) E782K, N986R, and R991L;
(pp) N968K, N986R, and R991L;
(qq) E782K, N968K, N986R, and R991L;
(rr) E782K, N986R, R991L, and R1015H;
(ss) N968K, N986R, R991L, and R1015H; and
(tt) E782K, N968K, N986R, R991L, and R1015H;

10. A variant SaCas9 protein comprising at least 95% amino acid sequence identity to the SaCas9 protein of any one of claims 1-9.

11. An isolated nucleic acid sequence encoding the variant SaCas9 protein of any one of claims 1-10.

12. A vector comprising the nucleic acid sequence of claim 11.

13. A system comprising:
(a) a guide RNA sequence that is complementary to a target genomic DNA sequence in a host cell, wherein the target genomic DNA sequence encodes at least one gene product; and
(b) a nucleic acid molecule comprising a nucleic acid sequence encoding the variant SaCas9 protein of any one of claims 1-10.

14. The system of claim 13, wherein the guide RNA sequence of (a) and the nucleic acid molecule of (b) are present in different vectors.

15. The system of claim 13, wherein the guide RNA sequence of (a) and the nucleic acid molecule of (b) are present in the same vector.

16. A system comprising:
(a) a guide RNA sequence that is complementary to a target genomic DNA sequence in a host cell, wherein the target genomic DNA sequence encodes at least one gene product; and
(b) the variant SaCas9 protein of any one of claims 1-10.

17. A method of altering a target genomic DNA sequence in a host cell, which method comprises contacting a host cell comprising a target genomic DNA sequence with the system of any one of claims 13-16, wherein:
(a) the guide RNA sequence is expressed in the host cell and binds to the target genomic DNA sequence in the host cell genome,
(b) the variant SaCas9 protein is expressed in the host cell and induces a double strand break in the target genomic DNA sequence, thereby altering the target genomic DNA sequence in the host cell.

18. The method of claim 17, wherein the host cell genome comprises a protospacer adjacent motif (PAM) comprising the nucleic acid sequence NNGRR[T/A/C/G] located adjacent to the target genomic DNA sequence, wherein “N” is guanine, adenine, thymine, or cytosine and “R” is guanine or adenine.

19. The method of claim 18, wherein the PAM comprises the nucleic acid sequence NNGRRT, NNGRRC, NNGRRA, or NNGRRG.

20. The method of any one of claims 17-19, wherein the target genomic DNA sequence encodes a protein.

21. The method of any one of claims 17-20, wherein the host cell is a mammalian cell.

22. The method of claim 21, wherein the host cell is a human cell.

23. Use of the system of any one of claims 13-16 for the alteration of a target DNA sequence in a host cell.

24. A method of generating a variant Cas9 protein with a desired PAM specificity, which method comprises:
(a) molecularly simulating binding of one or more mutant Cas9 proteins to a desired PAM;
(b) synthetically generating one or more mutant Cas9 proteins that bind to the desired PAM in the simulation of (a),
(c) expressing the one or more mutant Cas9 proteins in a host cell in combination with a guide RNA sequence that is complementary to a target DNA sequence in the host cell, wherein the host cell genome comprises the target DNA sequence and the desired PAM;
(d) measuring the cleavage activity of the one more mutant Cas9 proteins; and
(e) selecting one or more mutant Cas9 proteins which bind to the desired PAM and cleave the target DNA sequence, whereby a variant Cas9 with a desired PAM specificity is generated.

25. The method of claim 24, wherein molecularly simulating binding of one or more mutant Cas9 proteins to a desired PAM comprises free energy perturbation (FFP) calculations.
